(19) United States
(12) Patent Application Publication
(10) Pub. No.: US 2026/0367011 A1
(43) Pub. Date: Nov. 19, 2026

(54) A HYDROGEN SENSOR AND USE THEREOF

(71) Applicants: B. G. NEGEV TECHNOLOGIES AND APPLICATIONS LTD., AT BEN-GURION UNIVERSITY, Beer Sheva (IL); NUCLEAR RESEARCH CENTER NEGEV (NRCN), Beer Sheva (IL)

(72) Inventors: Oren Regev, Lehavim (IL); Yonatan Luzzatto, Beer-Sheva (IL); Amer Alatawna, Hurra (IL); Svetlana Pevzner, Beer Sheva (IL); Eli Peretz, Omer (IL)

(21) Appl. No.: 19/142,296

(22) PCT Filed: Dec. 21, 2023
(86) PCT No.: PCT/IB2023/063102

§ 371 Date: Jun. 23, 2025

(30) Foreign Application Priority Data
Dec. 21, 2022 (IL) 299369

(51) Int. Cl. G01N 21/78; G01N 21/77; G01N 33/00

(52) U.S. Cl. CPC G01N 21/783; G01N 33/005; G01N 2021/775

(57) ABSTRACT

A composite material-based visual sensor for the detection of hydrogen is disclosed, demonstrating reliable and robust multi-featured H2 detection abilities. The composite material comprises an H2 permeable polymeric matrix such as a silicon-containing polymeric matrix, a hydrogenation-susceptible organic compound such as a dye, and a hydrogenation reaction catalyst. Upon exposure of the sensor to H2, a hydrogenation reaction takes place, leading to a noticeable color change of the sensor within a few minutes, observable by the naked eye.

### FIELD OF THE INVENTION

The present disclosure relates to a color-sensitive device and use thereof, particularly but not exclusively, in detecting H₂ presence and reactivity.

### BACKGROUND

Hydrogen is a promising renewable energy source due to its lightweight abundance, non-toxicity, and high energy density. A global consumption of 625 million metric tons annually is predicted by 2050. Hydrogen is highly flammable and explosive and has the tendency to degrade metallic equipment such as pipes or tanks due to hydrogen embrittlement, consequently leading to leakage. Thus, hydrogen storage and transportation are challenging and expensive. Furthermore, hydrogen leakage and accumulation in closed environments, which are rather common due to their small molecular size, are highly dangerous and require the use of advanced hydrogen sensors and instrumentation that are not always available.

Although there are few technological solutions for delaying the degradation of metallic pipes and containers, monitoring potential hydrogen leakage is still considered a major challenge. Developing affordable and standalone hydrogen sensing systems that allow rapid detection is, therefore, essential. The above safety issues dictate that hydrogen is mainly produced near the point of use, creating an obstacle to its large-scale industrial applications.

Various hydrogen sensing technologies are in use nowadays, employing various types of hydrogen sensors based on different H₂ detection mechanisms; one example is hydrogen detection via electrical conductivity change that occurs in a given system when exposed to hydrogen gas. Metal-based visual sensors (e.g., gold nanoparticles and palladium films) chemically react with H₂, leading to a color change noticeable to the naked eye due to the formation of metal hydrides. For example, the transition of magnesium into magnesium hydride (MgH₂), which is associated with change of color upon Mg reduction, is used for hydrogen visual detection. Palladium-based microsensor is a further example, in which palladium is used as an H₂ sensor because it selectively absorbs hydrogen gas and forms the compound palladium hydride. However, palladium-based sensors have strong temperature dependency, which substantially prolongs their response time at very low temperatures. In addition, palladium sensors react with other gases such as carbon monoxide, sulfur dioxide, and hydrogen sulfide. While the detection time of metal-based visual sensors is rather short (seconds to minutes) under certain conditions, they tend to oxidize in atmospheric environments and are not specific to hydrogen. Moreover, they are rather expensive since costly metals constitute their main active reagents.

### SUMMARY

The present disclosure relates to the utilization of color changes of hydrogenation-susceptible organic compounds such as dyes as means for the visual detection of free H₂.

Disclosed herein is a composite material-based visual sensor for the detection of hydrogen presence, comprising an H₂ permeable polymeric matrix, a hydrogenation-susceptible (i.e., a reducible) organic compound, and a catalyst. Upon exposure of the sensor to H₂, a hydrogenation reaction takes place, leading to a noticeable and observable color change of the sensor within a few minutes. This color change is measured, and quantitative information, for example, kinetic parameters concerning, e.g., the hydrogenation rate and H₂ diffusion coefficient through the sensor, as well as temporal changes in H₂ concentrations in the vicinity of the sensor, are extracted.

The composite material-based H₂ sensor disclosed herein is an inexpensive, standalone hydrogen sensor demonstrating immediate detection of H₂ presence and high selectivity to hydrogen, unaffected by a humid environment.

In one aspect, the present disclosure relates to a composite material-based H₂ sensor comprising at least (i) an H₂ permeable polymeric matrix; (ii) a reducible organic molecule; and (iii) a catalyst. The H₂ sensor features at least one of the following properties:
(a) the reducible organic molecule is susceptible to a catalytic hydrogenation reaction upon exposure to H₂;
(b) the reducible organic molecule undergoes a detectable change of color and/or color shade upon hydrogenation;
(c) the sensor undergoes a detectable change of color and/or color shade upon exposure to a hydrogen-containing environment;
(d) a change of color is detectable and/or observable within seconds, minutes, or hours;
(e) the polymeric matrix is highly permeable to H₂. For example, the polymeric matrix affords penetration of H₂ into a depth of at least 200 μm within 1 hour; and
(f) the sensor is selective for hydrogenation reactions.

The change of color is observable by the naked eye. Alternatively, or additionally, the color change is detectable upon illumination with UV light.

The H₂ permeable polymeric matrix may comprise, for example, a silicon-containing polymer, a polyurethane polymer, or any combination thereof. In some embodiments, the composite material is based on a silicon-containing polymer such as, but not limited to, a polysiloxane, for example, polydimethylsiloxane (PDMS).

The reducible organic molecule or compound is often a conjugated molecule, for example, a dye. The catalyst may be a transitional metal, optionally Pd, Pt, Ni, Fe, Rh, or Ru, optionally supported on activated carbon, e.g., Pd/C, Pt/C, and the like.

An exemplary composite material-based H₂ sensor disclosed herein comprises PDMS as the polymeric matrix and 1,4 bis(phenyl ethynyl)benzene) (PEB) as the reducible organic compound. Upon reaction with H₂, this sensor changes its color shade from bright blue to dark blue as seen by the naked eye. Hydrogenation reaction in the composite material is promoted, e.g., by a Pd/C catalyst.

In a further aspect, the present disclosure relates to a method for detecting or monitoring the presence of hydrogen comprising the following steps:
(i) positioning a composite material-based H₂ sensor as described herein in an environment containing or suspected of containing free H₂; and
(ii) monitoring changes in the color and/or color shade of the sensor, whereby irreversible, stable, or progressive color and/or color shade change of the sensor is indicative of hydrogen reaction.

The hydrogen reaction rate in the composite material-based sensor may be determined. In some embodiments, the hydrogen reaction rate is determined by area analysis and/or mass analysis.

In yet another aspect, the present disclosure relates to a device or article comprising a composite material-based H₂ sensor as described herein, wherein, for example, the device or article is coated or is in contact with the composite material-based sensor.

### BRIEF DESCRIPTION OF THE DRAWINGS

Some embodiments are herein described by way of example only, with reference to the accompanying drawings. With specific reference now to the drawings in detail, it is stressed that the particulars shown are by way of example and for purposes of illustrative discussion of embodiments. In this regard, the description taken with the drawings makes apparent to those skilled in the art how embodiments may be practiced.

In the drawings:

FIGS. 1A-1B are light microscope images of a cross-section of a composite material-based H₂ sensor designed as a slab comprising a polydimethylsiloxane (PDMS) matrix and a mixture of 1,4-bis(phenylethynyl)benzene (PEB) and a metallic catalyst, following exposure to free hydrogen (P=500 mbar). 1A: a whole cross-section area, in which the dark area marks the hydrogenated part, and the bright area marks the non-hydrogenated part of the slab (delineated with a yellow dashed line). 1B: images of a specific sector in the sensor cross-section, following exposure to hydrogen for 1, 2, 5, and 14 hours;

FIG. 2 is a graph of the hydrogenated area calculated for images of FIG. 1B, using area analysis (AA) as a function of time;

FIG. 3 is a graph presenting the hydrogenation reaction progress in the cross-section of the slab shown in FIG. 1B as a function of time, evaluated using the area analysis (AA) calculation method (triangles) and mass analysis (dots). The blue and orange dash lines represent the logarithmic correlation for the obtained results;

FIG. 4 is a collection of images of H₂ sensor samples (PDMS-based composite material, comprising 10% (wt %) of a getter (mixture of PEB and a metal catalyst)) illuminated with 365 nm UV light. 4A: a sensor sample illuminated for 60 minutes. 4B: a further sensor sample wherein (a) the surface of the sensor was not exposed to hydrogen; (b) the surface of the sensor was exposed to H₂ for 14 hours (500 mbar); and (c) a cross-section of a sensor sample after partial hydrogenation (5 hr, 500 mbar);

FIG. 5 is a graph showing a collection of fluorescence emission spectra of an H₂ sensor (PDMS-getter comprising 10% (wt %) getter) illuminated with UV (365 nm UV light screening), following exposure to H₂ at 500 mbar for different time periods;

FIG. 6 is a collection of attenuated total reflectance (ATR)-Fourier transform infrared (FTIR) spectra obtained for H₂ sensor (PDMS-getter) samples exposed for 60 min to various gases: CO, CH₄, CO₂, H₂O, H₂+H₂O and H₂;

FIG. 7 is a calibration curve for the H₂ sensor (PDMS-getter), presenting the H₂ penetration depth per 1 hour of exposure (d) vs. H₂ pressure (P); and

FIG. 8 is an illustration of an H₂ sensor in the form of a slab utilized in a lacmus paper-like manner to provide information related to its H₂ exposure history: (a) and (b) the slab before and after H₂ exposure, respectively; (c) a cross-section of the slab; and (d) various optional color change penetration depths or color fronts (white arrow) in the cross-section, correlated to H₂ pressure and exposure time.

### DETAILED DESCRIPTION

Room temperature-vulcanizing silicone rubber loaded with unsaturated organic molecules has been used as a hydrogen scavenger in sealants such as O-rings. It was reported that after a long exposure time to hydrogen (days-weeks, at 130 mbar H₂), the silicon rubber underwent a color change. The need for a substantially shorter exposure time for color change to occur upon exposure to hydrogen remains an unmet need. Hence, a desired H₂ sensor should facilitate a faster hydrogen absorption rate, affording a higher conversion rate of the unsaturated organic molecules to their saturated state. A high absorption rate dictates a good hydrogen permeable hosting polymer.

The present disclosure relates to the utilization of the reaction of free hydrogen with a color-changing organic material contained within a permeable polymeric matrix for visual detection, even by the naked eye, and within minutes or hours of color changes yielded by hydrogenation or reduction of the organic material. Embodiments described herein utilize the hydrogenation reaction-based color change of organic molecules as an analytical tool for monitoring the presence and amounts of free H₂.

The present inventors designed and successfully constructed devices, practical, inexpensive, and simple visual sensors for hydrogen reactivity. The devices disclosed herein are composite material-based sensors comprising a permeable polymeric matrix (e.g., a polydimethylsiloxane (PDMS) matrix) loaded with a mixture of unsaturated, reduceable organic molecules (e.g., diphenyl ethylbenzene (DEB) (also known as 1,4-bis(phenylethynyl)benzene (PEB)) and a metallic catalyst (e.g., Pd/C). This mixture is termed “getter”, and the entire composite material (including the permeable matrix) is termed “polymer-getter” (e.g., PDMS-getter). Exemplary devices are configured like slabs, chips, or ribbons.

Hydrogen, arriving at the surface of the sensor, travels through the composite material matrix and catalytically reacts with reduceable organic molecules, yielding fully or partially saturated organic molecules. For example, when the reduceable organic molecule is PEB, this chemical reaction leads to a noticeable color shade change of the PDMS-getter from bright to dark and enables the detection of hydrogen presence.

The term “composite material”, as used herein, refers to a unified combination of two or more distinct materials with an intended purpose to achieve desired properties. In the context of the present disclosure, the term “composite material” refers to the material formed by the unified combination of a polymer, hydrogenation-susceptible organic molecules and a catalyst that facilitates hydrogenation or reduction of the organic molecules.

The composite material-based H₂ sensors disclosed herein yield a noticeable and fast color change upon exposure to hydrogen and, therefore, can be employed as a standalone hydrogen sensor.

Visual, qualitative assessment of the reduction reaction can be quantified, for example, by conversion calculation performed using means such as area analysis (AA) of sections of the composite that have undergone color or color shade change. As further defined and demonstrated in the examples disclosed herein, the color-changing area enables the estimation of the polymer composite's hydrogenation rate and H₂ diffusion coefficient.

In one aspect, the present disclosure relates to a composite material-based sensor for the detection of hydrogen reaction comprising:
(i) an H₂ permeable polymeric matrix;
(ii) a reducible organic compound; and
(iii) a catalyst.

For brevity, a contemplated composite material-based sensor for detecting the hydrogen reaction is referred to herein as an “H₂ sensor”.

In some embodiments, the H₂ sensor features at least one of:
(a) the reducible organic compound is susceptible to a catalytic hydrogenation reaction upon exposure to H₂;
(b) the reducible organic compound undergoes a detectable change of color and/or color shade upon hydrogenation;
(c) the sensor undergoes a detectable change of color and/or color shade upon exposure to a hydrogen-containing environment;
(d) a change of color is detectable and/or observable within seconds, minutes, or hours;
(e) the polymeric matrix is highly permeable to H₂; and
(f) the sensor is highly selective to H₂, even in an environment containing H₂O vapors (humid environment) and/or other gases.

In some embodiments, the polymeric matrix is transparent or translucent.

A critical feature of a contemplated H₂ sensor is the high permeability of the polymeric matrix to H₂ for the sensor to operate efficiently. Permeability to hydrogen allows the absorption of H₂ into the matrix and facilitates the hydrogenation reaction. Moreover, the sensor is adapted to detect the presence of hydrogen in short time frames, i.e., minutes or hours, and better permeability may result in shorter detection times.

The H₂ sensor undergoes, in most cases, an irreversible hydrogenation reaction upon exposure to H₂ in its environment. Yet, in some embodiments, the sensor can be reused by stripping it of its hydrogen content and regenerating (oxidizing, dehydrogenating) the reduced compound to its original form.

In some embodiments, the H₂ sensor may react with free H₂ at relatively low pressures.

In some embodiments, the change of color or color shade in the H₂ sensor is readily observable to the naked eye. Additionally, or alternatively, the change of color or color shade is observable upon exposure to a certain illumination and/or radiation. In some embodiments, the change of color or color shade may be assessed by illumination with UV light. More precise color measurements can be performed using conventional colorimetric equipment or means.

The hydrogenation reaction of an H₂ sensor described herein may become observable within seconds, minutes, or hours after exposure to free H₂. In some embodiments, a change of color may be observed, e.g., within 1, 2, 3, 4, 5, 6, 7, 8, 9, 10, 12, 15, 18, 20, 24, 26, 28, 30 min or longer. In some embodiments, a color change of the sensor is observed within 2-10 min, for example, within 2-5 min after exposing the sensor to free H₂.

The relative amounts (e.g., % weight) of each of components (i)-(iii) of the H₂ sensor may vary depending on at least one of: (i) the type and properties of the polymeric matrix; (ii) the nature and sensitivity of the reducible organic compound; (iii) the type and reactivity of the catalyst; and/or (iv) the intended use of the sensor. For example, for an intended exposure of the sensor to low H₂ pressure, the relative amount of the reducible organic compound and/or the catalyst in the matrix may be higher compared to the H₂ sensor intended for use at high H₂ pressures to confer better or increased sensitivity to the sensor.

As a rule, the sensitivity of the sensor to H₂ reactivity may be increased by one or more of: increasing the amounts of reducible organic compound and/or the catalyst; increasing the reactivity of the reducible organic compound; and/or increasing the permeability of the polymeric matrix.

In some embodiments, the organic molecules and the catalyst (namely, the getter) are about 8% or more of the total weight of the composite material. For example, the getter may constitute about 8%, 9%, 10%, 12%, 14%, 15%, 18%, 20%, 22%, 25%, 28% by weight (% wt) or more of the composite material.

In some embodiments, the visual effect is achieved with less that 8% getter of total composite weight, e.g., 7%, 6% or less. In some embodiments, the visual effect is achieved with less that 10% getter of total composite weight.

The organic molecules:catalyst ratio is usually in the range of from 1:1 to 3:1 for example, 1:1, 1.5:1, 2:1, 2.5:1 or 3:1.

The organic, reducible compound or the getter may be mixed and molded together with the matrix to provide a homogeneous composite material.

In some embodiments, the H₂ permeable polymeric matrix comprises a silicon-containing polymer, a polyurethane polymer, or any combination of copolymers thereof.

The term “silicon-containing polymer”, as used herein, refers to a polymer comprising silicon atoms in its backbone and/or side chains.

In some embodiments, the silicon-containing polymer is silicone or silicone-based polymer. The terms “silicone”, “silicone-based polymer”, “polysiloxane” and “siloxane polymer”, as used herein, are interchangeable and refer to a synthetic polymer with a silicon-oxygen backbone chain ( . . . —Si—O—Si—O—Si—O— . . . ), and organic groups attached to the silicon atoms by C—Si bond. For example, polysiloxanes encompass silicone-based polymers comprising repeating siloxane groups (Si—O—Si) in the backbone, side chains, or crosslinks.

In the context of the present disclosure, silicone-based polymers and oligomers may be represented by the general formula [—Si(R₂)—O-]ₙ, wherein n is at least 10, and each R (residue, moiety), independently, represents H or an organic group attached to the silicone backbone, which may be a hydrocarbyl, for example, methyl, ethenyl (vinyl) or phenyl, or a substituted hydrocarbyl, for example, an alkyl or phenyl substituted by hydroxy, alkoxy (alkyl-OH) and/or a reactive residue such as amine, epoxy, isocyanate, vinyl, halide (e.g., trifluoropropyl), urea and/or oxysilane as well as other functional groups that enable reactions with organic functionalities. For example, in organic reactive silicones, R may be an alkoxy and undergo hydrolytic polycondensation reactions.

Different silicone-based polymers are distinguished by systematic names that are based on the monomeric unit (before polymerization), presented herein as R₄Si. For example, when all Rs are H, the monomer R₄Si is referred to herein as “silane”, and when all Rs are alkoxy groups or hydroxy groups, the monomer (RO)₄Si or (HO)₄Si, respectively, are both referred to herein as “oxysilane”. Silicone-based polymers comprising polyoxysilanes are represented by the formula [—Si(RO)₂—O-]ₙ.

Non-limiting, exemplary polysiloxanes that can make the polymeric matrix in accordance with embodiments described herein include polydimethylsiloxane (PDMS; with repeating unit (monomer) [—Si(CH₃)₂—O-]ₙ), polymethylhydrogensiloxane (PMHS; repeating unit [—Si(CH₃)(H)—O-]ₙ), polydiethylsiloxane (PDES; repeating unit [—Si(C₂H₅)₂—O-]ₙ), polyphenylmethylsiloxane (PMPS; repeating unit [—Si(CH₃)(C₆H₅)—O-]ₙ), polydiphenylsiloxane (PDPS; repeating unit [—Si(C₆H₅)₂—O-]ₙ), poly(di-n-propylsiloxane) (repeating unit [—Si(C₃H₇)₂—O-]ₙ), polyditolylsiloxane (repeating unit [—Si(CH₃—C₆H₅)₂—O-]ₙ), and polyphenyltolylsiloxane (repeating unit [—Si(C₆H₅)(CH₃—C₆H₅)—O-]ₙ). Further exemplary polysiloxanes are ones having unusually long side chain residues, for example, C₆H₁₃, C₁₆H₃₃, and C₁₈H₃₇ and/or branched side chains such as —CH(CH₃—(CH₂)ₘ—CH₃. Yet further exemplary polysiloxanes are ones having methoxy-substituted aromatic fragments as one of the two residues (R) in the monomeric unit. Other silicone-based polymers include optically active groups, the simplest example being the secondary butyl group —CH(CH₃)(C₂H₅). Yet further exemplary silicone-based polymers include one having one or two phenylethenyl groups, cyclic siloxane groups, and/or phenylacetylene groups.

In some embodiments, the silicone-based polymer is polydimethylsiloxane (PDMS), also known as dimethylpolysiloxane or dimethicone. In some embodiments, the PDMS is a vinyl-terminated dimethylpolysiloxane.

Further silicon-containing polymers useful for forming a contemplated polymeric matrix, in accordance with some embodiments described herein, include:
(i) silalkylene polymers, herein referring to silicon-containing polymers in which methylene groups replace the oxygen atoms of polysiloxanes in the backbone. Poly(dimethylsilmethylene) having the repeating unit [—Si(CH₃)₂—CH₂-]ₓ is an example;
(ii) silarylene polymers, herein referring to silicon-containing polymers in which aryl groups replace the oxygen atoms of polysiloxanes, for example, in poly(dimethyldiphenylsilylenemethylene) (repeating unit [—Si(CH₃)₂CH₂Si(C₆H₅)₂-]ₓ. Other aryl substituents are, for example, tolyl groups;
(iii) siloxane-silarylene polymers, in which a silarylene group, for example, silphenylene group [—Si(CH₃)₂—C₆H₄—], is inserted into the backbone of the polysiloxane repeat unit to give [—Si(CH₃)₂—C₆H₄—Si(CH₃)₂O-]ₓ, or [—Si(CH₃)₂—O—Si(CH₃)₂—C₆H₄-]ₓ, in which the phenylenes are either ortho, meta or para substituted. A specific example is poly(tetramethyl-p-silphenylene-siloxane);
(iv) sesquisiloxane polymers having a ladder structure;
(v) polysilanes and polysilylenes, [—SiR₂-]ₓ, in which the Rs may be the same or different substituted or unsubstituted hydrocarbyl;
(vi) polysilazanes, [—SiRR′—NR′-]ₓ, wherein R and R′, each independently, may be the same or different substituted or unsubstituted hydrocarbyl; and
(vii) random and block copolymers, and any combination of two or more of the silicon-containing polymers described herein.

x is at least 10.

In some embodiments, the H₂ permeable polymeric matrix comprises a polyurethane polymer.

Polyurethane (PUR or PU), as used herein, refers to a class of polymers composed of organic units joined by urethane (—HN—C(═O)—O—) and/or carbamate (—HN—C(═O)—O—CH₂CH₂—) links. Polyurethanes may be produced from a wide range of starting materials and, therefore, present different chemical structures, leading to many different applications. As a solid, polyurethane is a plastic-like substance having an open-cellular structure, which is a foam.

The term “reducible organic compound”, as used herein, refers to a molecule that undergoes an increase in the number of carbon-hydrogen bonds and a reduction in carbon-carbon and/or carbon-heteroatom bonds upon reaction with H₂. Reduction of an organic molecule is interchangeably referred to herein as “hydrogenation of the organic molecule” or “hydrogenation of the reducible organic compound”.

In some embodiments, the reducible organic compound is a molecule that contains at least two carbon-carbon double and/or triple bonds. Such molecule is also referred to herein as “unsaturated molecule” and includes, for example, a hydrocarbon selected from an alkene, alkyne or an aromatic hydrocarbon, optionally substituted by a functional group such as, but not limited to, an alkene, ether, amine sulfides, phosphate ester, aldehyde, keto, imine, epoxide, and carboxylic acid.

In some embodiments, the unsaturated molecule is a conjugated molecule, namely, a molecule containing two or more multiple (i.e., double or triple) bonds alternating with single bonds. An extended conjugated system exists over a long series of atoms (e.g., C═C—C═C, C═C—C═N, C═C—C═O, C═C—C—Ar, wherein Ar is aryl, etc.).

In some embodiments, the reducible molecule is a dye, for example, an organic dye. Hydrogenation of a dye may result in a change of color and/or color shade. For example, a colored dye can become colorless upon hydrogenation, e.g., red, blue or purple dye molecules may become colorless, or the color may switch from, e.g., blue to black, or a dark color of a dye may become brighter, and the like.

Reducible dye compounds or molecules useful for the purpose of the present disclosure include but are not limited to, diphenyl ethylbenzene (PEB; 1,4 bis(phenyl ethynyl)benzene), diphenyl butadiyne, diphenyl acetylene, polybutadiene, or polyphenylethynylbenzene, and any combination thereof.

In some embodiments, hydrogenation of the reducible molecule, e.g., a dye, is effected in the presence of a catalyst. Catalytic hydrogenation is utilized, at least in some embodiments described herein, for promoting irreversible color switching of dyes to facilitate detection or observation of the hydrogenation reaction.

When an organic molecule is being oxidized or reduced, a catalyst, which is a redox agent, is being reduced or oxidized, respectively, as oxidation and reduction always occur in tandem. The choice of catalyst applied in the hydrogenation of the unsaturated reducible molecules, e.g., an organic dye as disclosed herein, depends on several factors, such as the reactivity of the substrate and the environmental conditions (pressure, temperature, and the like). In addition, sometimes, the nature of the catalyst's support plays a key role in its catalytic performance. For a given reaction, the activity, selectivity, and stability of the catalyst may be improved by the use of appropriate support. Hydrogenation catalysts that may be employed in accordance with the present disclosure are transitional metals such as, but are not limited to, palladium (Pd), platinum (Pt), nickel (Ni), rhodium (Rh), iron (Fe) and/or ruthenium (Ru).

In some embodiments, the metal catalyst is Pd on activated carbon (Pd/C), or on other high surface area support.

In some embodiments, a composite material disclosed herein comprises polydimethylsiloxane (PDMS) as the polymeric matrix and PEB as the reducible organic compound. Upon reaction with H₂, this composite material changes its color shade from bright blue to dark blue. Hydrogenation reaction in the composite material may be promoted by a Pd/C catalyst.

The hydrogen sensor described herein is useful in detecting the presence of hydrogen. As such, it complies with some basic requirements for sensitivity, reliability, performance, and/or response time:
(i) the functionality of the disclosed H₂ sensor is easily verifiable;
(ii) the sensor may function in an ambient air or gas environment; and/or
(iii) the sensor is resistant to environmental interferences.

The H₂ sensor disclosed herein is useful for qualitative and/or quantitative monitoring of free H₂ in its environment. First, a qualitative assessment of free H₂ presence is enabled based on visualization of color/color shade changes in the sensor. Then, visual and/or physical changes in the sensor can be measured and provide valuable information such as H₂ concentration, exposure time, and the like.

In another aspect, the present disclosure relates to a method for detecting or monitoring a hydrogen reaction comprising:
(i) positioning a composite material-based H₂ sensor as defined herein in an environment containing or suspected of containing free H₂;
(ii) monitoring changes in the color and/or color shade of the sensor, whereby irreversible stable or progressive color and/or color shade change of the sensor is indicative of hydrogen reaction; and
(iii) optionally, determining the hydrogen reaction rate in the composite material.

Means by which hydrogen reaction rate can be determined include, for example, area analysis and mass analysis, as further described in the Examples section herein. Other or alternative means include chemical analysis such as gas chromatography of organic materials extracted from the matrix, e.g., reduced compounds.

Various H₂ sensors are known in the art. For example, U.S. Patent application Publication Nos. 2007/251822 and 2022/0276176 disclose visual hydrogen sensors. US 2007/251822 discloses a sensor composed of a transition metal oxide such as tungsten oxide or yttrium oxide that, when exposed to hydrogen, reduces and changes its color, a catalyst such as platinum, palladium, rhodium, nickel, etc., and a molecular diffusion barrier that allows permeable diffusion of hydrogen, optionally made of a polymer such as polysiloxane or polyurethane. The hydrogen sensor is supported on a substrate. US 2022/0276176 discloses a hydrogen sensor comprising a color-changeable layer disposed on a substrate that supports the hydrogen sensor and is made of an oxide semiconductor material such as tungsten oxide. When the oxide semiconductor material reacts with hydrogen ions or hydrogen atoms, the color thereof changes. The hydrogen sensor further comprises a catalyst layer disposed on a surface of the color-changeable layer (e.g., palladium (Pd), platinum (Pt)) and a protective layer made of a polymer material such as polyvinyl butyral (PVB), wherein the polymer material allows hydrogen molecules to pass through. The hydrogen sensors disclosed in these publications are multi-layered sensors. In this configuration, each layer is separated from the other and possesses different functionality. To be noted, while the prior art sensors use polymer-based materials as a solution for protecting the homogeneous reactive layer from air, in the composite-material-based H₂ sensor disclosed herein, the polymer matrix is an integral part of the reactive function of the sensor and the active agents do not need protection. This is a different discipline originating from the composite material field, and implementation thereof requires specific knowledge.

Furthermore, the organic reducible material-based H₂ sensors disclosed herein are considerably less expensive compared to known inorganic, metal-based sensors. Metal-based materials are also less environmental (more pollutant) compared to organic material-based sensors. Moreover, the mechanism that leads to a color change is completely different: the H₂ sensors disclosed herein are based on catalytic hydrogenation that occurs in a polymeric medium, which is counterintuitive to known mechanisms of H₂ detection.

The H₂ sensor may be produced or fabricated in various forms, depending on its intended use and the nature of the polymeric matrix used. Non-limiting forms include a slab, chip, coating layer, pellet, gel, or tape.

For example, the H₂ detector may be utilized as a leak detector. As such, it can be applied as a color-changing self-fusing wrap or coating tape designed to detect hydrogen gas leaks in fuel cells, transmission, storage and generation facilities. In the event of a leak due to vibration or seal failure, the tape changes its color upon contact with hydrogen gas. Inspection teams can use this low-cost hydrogen leak detector to accurately identify leak locations and begin repairs, expediting equipment restoration and improving safety.

A device or article coated with or attached or connected to an H₂ sensor described herein may be, for example, the inlet, outlet, or connection points of pipes of tanks or reservoirs containing H₂.

The terms “comprise”, “comprising”, “includes”, “including”, “having” and their conjugates mean “including but not limited to”.

The term “consisting of” means “including and limited to”

As used herein, the singular form “a”, “an” and “the” include plural references unless the context clearly dictates otherwise. For example, the term “a compound” or “at least one compound” may include a plurality of compounds, including mixtures thereof.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Various embodiments and aspects, as delineated hereinabove and as claimed in the claims section below, find experimental support in the following examples.

### EXAMPLES

Reference is now made to the following examples, which, together with the above descriptions, illustrate some embodiments in a non-limiting fashion.

### Materials and Methods

### Materials

Polydimethylsiloxane vinyldimethylsiloxy terminated (Alfa Aesar, Mw=6000 g mol⁻¹, resin), tetrakis(dimethylsiloxy)silane 97% (Alfa Aesar, Mw=324.7 g mol⁻¹, cross-linker), cis-dichlorobis(diethylsulfide)platinum(II), 99% (Strem Ltd., catalyst), 1,4-bis(phenylethynyl)benzene (PEB) (was synthesized according to previously described methods (S. Havens et al., J. Polym. Sci., Polym. Chem. Ed., 1981, 19, 1349-1356; Pri-Bar and J. E. Koresh, J. Mol. Catal. A: Chem., 2003, 206, 313-318)), palladium on carbon catalyst (Pd/C, Sigma-Aldrich-7599250G, 5 wt %), toluene (Daejung Chemicals), were used as received.

### Sensor Production

Getter preparation. The getter powder PEB:Pd/C in a ratio of 2:1 wt/wt were prepared by ball-milling (Fritsch Pulverisette 6, 14 agate balls, 10 mm diameter, operated at 600 rpm for 15 min). Powder:balls weight ratio was 1.5:1.

Polydimethylsiloxane (PDMS) polymer preparation. A polymer resin, cross-linker (24:1 wt/wt), and 10 ml of a catalyst solution (2 vol % of catalyst in toluene) were mixed in a Thinky mixer (ARE-100, 2000 rpm) for 5 min in the presence of 1 zirconia ball (10 mm in diameter). The total weight was 5 g. The mixture was cast in plastic molds (25×8×1.5 mm³) and cured for 24 hours at room temperature (RT). Samples were cut into small rectangular samples weighing 130-170 mg for a hydrogen absorption test.

Preparation of composites. The PDMS-getter composite material was prepared using a similar procedure to the PDMS polymer preparation described above, where the getter powder (25 wt %) was added to the mixture before Thinky mixing.

### Gravimetric Analysis

The H₂ sorption experiments were performed in an Intelligent Gravimetric Analyzer (IGA 003, Hiden Isochema). The IGA-003 is a dynamic gas sorption analyzer incorporating multiple controllable inlets with combined pressure and flow control, and integrated mass spectrometry. The IGA-003 measures changes in sample mass as a function of temperature, pressure and gas composition. The unique IGA method analyzes real-time gravimetric data to determine kinetic parameters and simultaneously predict sorption equilibrium. This system was used to measure the total hydrogen sorption in the polymer and its kinetics (from which the diffusion and hydrogenation kinetic coefficients were calculated, vide infra). During the experiment, a rectangular sample (150±20 mg) was placed in a holder inside a stainless-steel reactor, where degassing was performed at 10⁻³ mbar and 323 K for 2 hours. This procedure was followed by overnight degassing at room temperature before exposing the sample to hydrogen (500 mbar). The IGA system monitored the weight gain (accuracy of about 0.1 μg) of the sample over time.

### Light Microscopy

The samples were imaged by using a light microscope (Leica M165 FC) and a digital microscope color camera (Flexacam C1) combined with white light (Ring illuminator, Leica LED5000 RL). The surface and the cross-section of each sample were examined before and after the hydrogenation process in the IGA.

### UV Torch Illumination and Emitted Fluorescence Measurement

The composite samples were screened with a UV torchlight (TROTEC, UV Torchlight 15F, 365 nm, 3 Watt). This lightweight LED torch provides maximum UV-A performance immediately after switch-on and is especially suited for quick inspections or checks of poorly accessible areas. The high spotlight radiation intensity of the UV torchlight enables the achievement of a high fluorescence stimulation, thereby, even minor tracers are clearly visible in the daylight. This torch improved hydrogen detection by enhancing the contrast between reacted and unreacted areas.

Fluorescence emitted following UV illumination was measured using Fluorolog®-3 (Horiba). This instrument enables a spectral analysis of different fluorophores in liquid and solid samples.

### FTIR Spectroscopy

Attenuated total reflectance (ATR) is a sampling methodology that enables the direct examination of solid or liquid samples without further preparation. It utilizes total internal reflection to generate an evanescent wave that penetrates the sample, providing valuable molecular information. ATR is often used with Fourier transform infrared (FTIR) spectroscopy (ATR-FTIR spectroscopy) as it enables solids and liquid samples to be analyzed neatly—simplifying the measurement of substances. ATR-FTIR spectroscopy is utilized for tasks such as identifying chemical compounds, studying molecular structures, examining surface properties, analyzing polymers, investigating biomolecules, monitoring chemical reactions, and assessing the composition of materials.

Contrary to transmission, the measurement path length is independent of the thickness of the sample. ATR is an internal reflection-based method, and the sample path length is dependent on the depth of penetration of the infrared energy into the sample.

The composite material-based H₂ sensor samples were analyzed using ATR-FTIR spectroscopy (Nicolet™ iS™ 5, Thermo Fischer) operated in the spectral range of 500-4000 cm⁻¹ with 32 scans and a resolution of 8 cm⁻¹. Background substruction and baseline correction were performed using Omnic Specta (Thermo Fischer).

### Example 1

### Visual Analysis of Hydrogen Reaction and Diffusion in the H₂ Sensor

Visual analysis of the course of reduction reaction taking place on the surface and inside a composite-material-based H₂ sensor formed as a slab and consisting of a polydimethylsiloxane (PDMS) matrix loaded with a mixture of 1,4-bis(phenylethynyl)benzene (PEB) and a metallic catalyst (PDMS-getter), was assessed over time. The color change of a PDMS-getter composite upon exposure to H₂ was investigated by the naked eye.

The sensor was exposed to hydrogen at a partial pressure of P=500 mbar for a period of 1, 2, 5, and 14 hours. The results are shown in FIGS. 1A-1B.

Upon exposure to hydrogen, the sensor showed a noticeable color change from bright to dark, without any known side reactions. FIG. 1B shows the color change in a whole cross-section of the slab. The non-hydrogenated part/area of the sensor is delineated with a yellow dashed line.

This change is attributed directly to the hydrogenation of PEB since it breaks the conjugation of the molecule and, therefore, leads to a change in its light emission. The hydrogenation reaction between PEB and four molecules of H₂ (on the surface of a Pd/C catalyst) that forms fully saturated 8H-PEB is shown in Scheme 1:

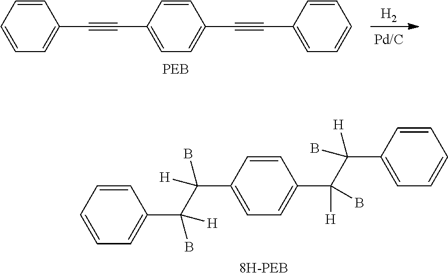

This phenomenon allowed the detection of hydrogen presence by the naked eye.

To determine whether the change in the dark area correlates with the progress of the reaction, an area analysis (AA) was performed, and the ratio

$$\frac{dark area}{total cross - sectio area}$$

was calculated and compared to the conversion % of PEB to 8H-PEB. The amount of 8H-PEB was assessed by mass analysis (MA) using the intelligent gravimetric analyzer (IGA) technology described in Materials and Methods.

Examination of the composite's cross-section (performed in a light microscope) revealed a hydrogenated front (black for hydrogenated and blue for unreacted material, Error! Reference source not found.), which increased as a function of the exposure time. Surprisingly, the penetration depth was 200 μm after only 1 hour. Comparable penetration in known sensors was obtainable only within 3 weeks or longer.

### Example 2

### Hydrogen Diffusion Quantification

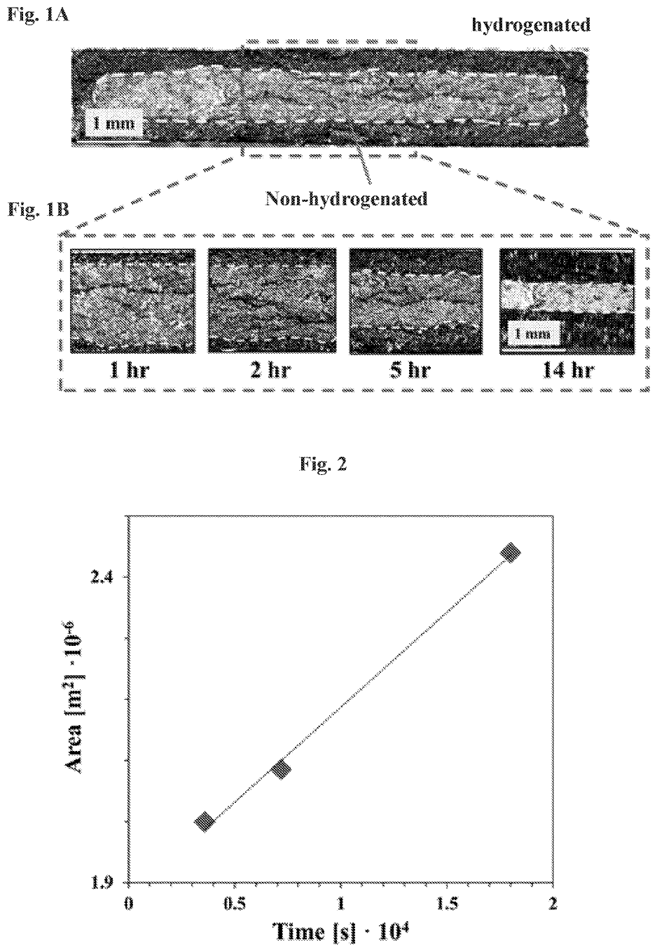

The area analysis method was expanded for quantifying the hydrogen diffusion through the PDMS-getter sensor of Example 1. The dark area change was monitored over time, and the change in the dark area vs. time was plotted. A linear correlation between the dark (hydrogenated) area and the diffusion time has been obtained. The appearance of a dark area indicated that the hydrogenation process has started, and since hydrogen immediately reacts with PEB molecules on the sensor surface, the progression of the dark area was assumed to be equivalent to the progression of hydrogen. The diffusion coefficient was extracted from the slope of the graph. Analysis of dark area change over time for a cross-section of the PDMS-based sensor is shown in FIG. 2. The slope of the linear graph, i.e., the diffusion coefficient (D), was calculated using equation 1:

$$slope = D = \frac{ΔArea}{Δ t}$$

where:
ΔArea—the change in the dark area; and
Δt—the exposure time to hydrogen.

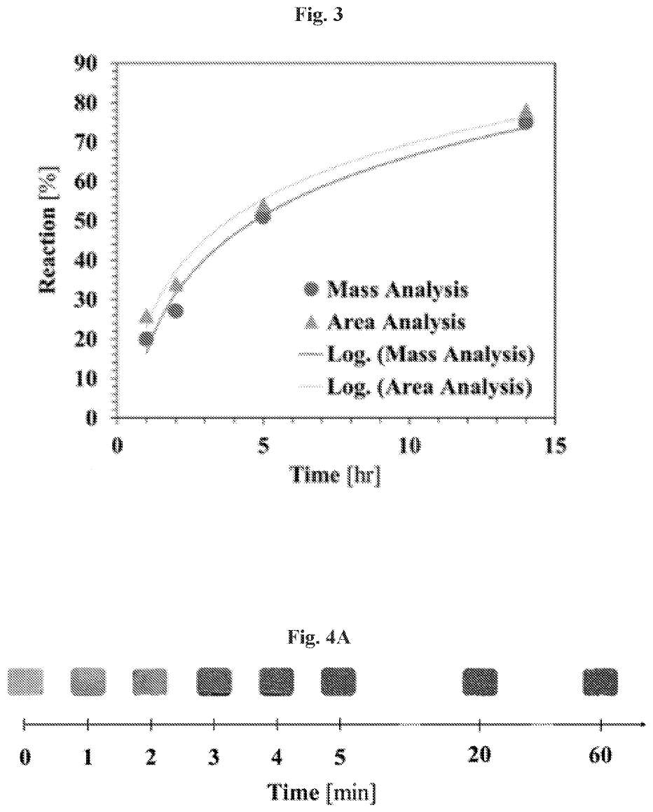

To validate the creditability of the AA, the results obtained from the AA, i.e., the diffusion parameter, were compared to mass analysis (MA) results. In the latter method, the reduction reaction was estimated by monitoring the weight gain of the same sensor samples in a hydrogenous environment. The MA was performed in an Intelligent Gravimetric Analyzer (IGA), which can detect slight weight changes due to hydrogen's chemical absorption. The comparison of MA and AA results for the four sensor cross-section samples is shown in FIG. 1B, and the logarithmic correlation for the obtained results is presented in FIG. 3.

As seen in FIG. 3, The AA and MA results were aligned. These results prove that the PDMS-getter slab can serve as a sensitive H₂ sensor for visual detection of hydrogen presence and reactivity. The results further suggest that quantification of the color change of the sensor provides a reliable estimation of hydrogen's reaction rate and diffusion coefficient.

### Example 3

### Fluorescence-Based Detection of H₂ Sensor Hydrogenation

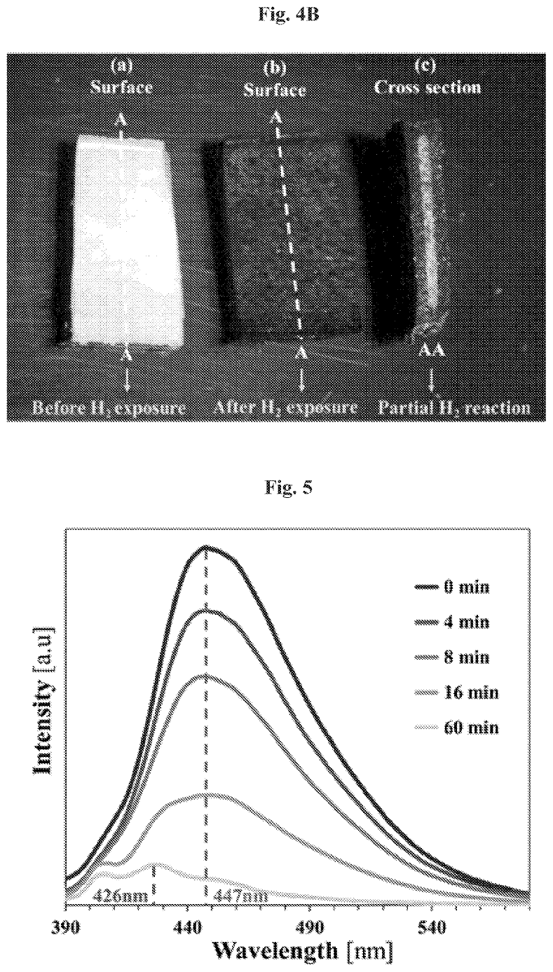

The H₂ sensor's fluorescence following illumination with UV light was observed visually by the naked eye and measured spectrally, and the change of color/fluorescence as a function of H₂ exposure time was assessed. The sensor was a PDMS-getter composite comprising a 10% (% w) PEB-based getter. The results are shown in FIGS. 4A-4B and FIG. 5.

FIG. 4A shows the gradual change of color of a sensor sample exposed to H₂ for 60 min, upon illumination by 365 nm UV light. FIG. 4B shows the UV-illuminated surface of the sensor before exposure to H₂ (a) and following 14 hours of H₂ exposure (b). A cross-section of the sensor following a 5-hour exposure to H₂ is also shown (c). When not exposed to hydrogen, the sensor emitted bright blue light when illuminated by a UV torch (365 nm). However, upon exposure to hydrogen (500 mbar), the reacted regions underwent a color change from blue to purple, as seen both on the sensor surface and in its cross-section.

The fluorescence intensity and wavelength correlated with H₂ exposure time. As seen in FIG. 5, the fluorescence intensity significantly decreased after 4 minutes of H₂ exposure. Moreover, the peak of maximum fluorescence intensity shifted from 447 to 426 nm due to hydrogenation, in line with the naked-eye observation (Error! Reference source not found.4A-4B). The decrease in fluorescence intensity is expected since, during H₂ exposure, the C—C bonds of the dye PEB gradually shift from an unsaturated state (conjugated triple bonds) to a saturated one.

These results demonstrate that screening the H₂ sensor with UV light provides a further means for enhanced hydrogen detection abilities due to the ability of UV illumination to detect rapid, noticeable color/fluorescence change following only 2-3 minutes of H₂ exposure.

### Example 4

### Specificity Tests

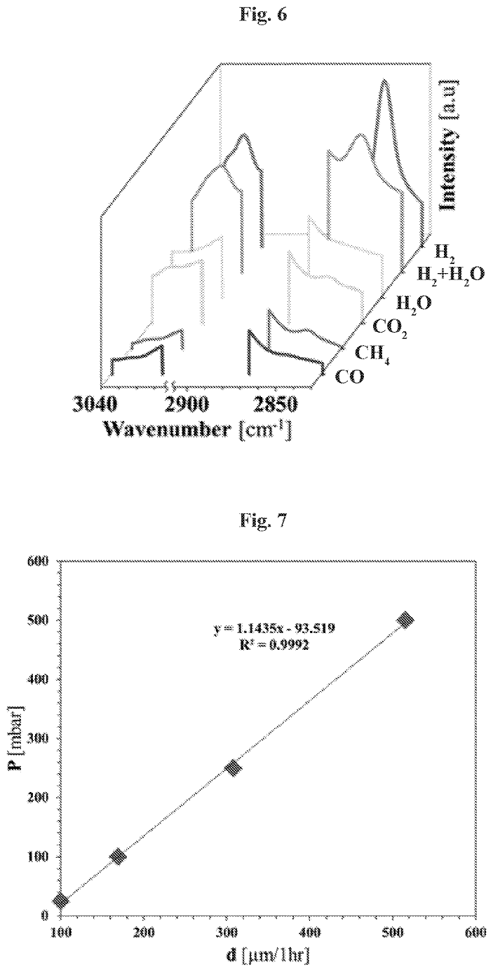

An essential feature of a sensor is its specificity, namely, that it indicates a change only when exposed to the analyte of interest. For determining the sensor's specificity to hydrogen, a chemical analysis (ATR-FTIR) was performed to determine whether the I-bonds of the PEB molecule reacted and whether the conjugation broke, using the PDMS-getter composite material of Example 1. For this purpose, the sensor was exposed to various gases commonly present in the atmosphere, such as CO₂, CO, CH₄, H₂O (vapor), and H₂+H₂O, each for at least 60 min. The results are shown in FIG. 6.

First, a PDMS-getter sample was subject to FTIR analysis before exposure to H₂ and then after being exposed to H₂ for 60 min. Changes in the chemical conjugation state following hydrogenation of PEB were evaluated based on the differences in the FTIR spectra of the sensor sample before and after H₂ exposure. As seen in FIG. 6, following H₂ exposure, significant peaks were observed at wavelengths 2800-2860 and 3000-3050 cm⁻¹, characterizing semi- or fully saturated bonds, namely, double C—H bonds and single bonds, respectively. Sensor samples not exposed to H₂, showed non-saturated bonds signature (results not shown).

Then, PDMS-getter samples were exposed at 500 mbar for 60 min to CO₂, CO, CH₄, and H₂O gases, and the same FTIR-based analysis was performed. Furthermore, the effect of humidity (H₂O vapor) on the hydrogenation of PEB was assessed by exposing the sensor to a gaseous H₂/H₂O mixture. The FTIR spectra obtained for sensor samples exposed to each of these gases were compared to those obtained following 60 min of exposure to H₂ (FIG. 6).

Chemical analysis of the results clearly indicates that a reaction with PEB in the sensor occurred only after H₂ exposure, while none of the other gases caused chemical changes in the PDMS-getter samples. Moreover, chemical analysis of the sensor sample exposed to an H₂/H₂O mixture showed a similar FTIR signature as exposure to H₂, indicating that a humid environment did not have a major impact on the performance of the sensor.

These results demonstrate the high specificity of the sensor to H₂ gas and prove that it can serve as a reliable and robust sensor in an atmospheric environment.

### Example 5

### Calibration of the H₂ Exposure Parameters

The visual change in color/color shade observed by the naked eye and/or upon UV illumination of the H₂ sensor exposed to H₂ under varying pressures and/or durations may provide valuable information about the hydrogen exposure history, in an analogy to a lacmus paper. Determining the penetration depth of hydrogen at any given time provides information about the H₂ environment's conditions.

Exposing the sensor to different H₂ pressures yielded different hydrogen penetration depths. Thus, H₂ penetration depth could be correlated with the partial pressure of hydrogen at a given time. Based on such observations, the calibration curve shown in FIG. 7 was obtained, presenting penetration depth per 1 hour of H₂ exposure (d) vs. H₂ partial pressure.

From this calibration curve, the partial pressure of H₂ in a given environment can be extracted.

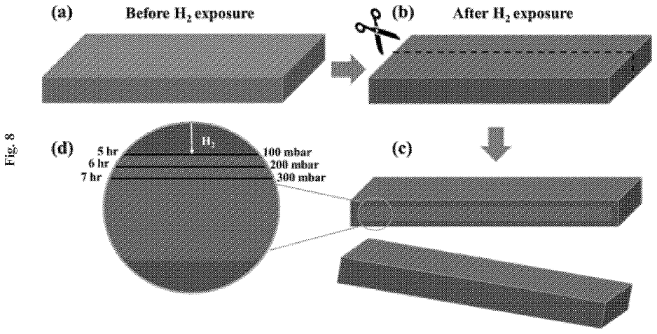

Hence, in addition to its ability to detect hydrogen, the H₂ sensor can also provide the H₂ exposure history, whereby both the partial pressure of hydrogen in a given environment and the hydrogen exposure period can be evaluated. A schematic presentation of the sensor formed as a slab and functioning as a lacmus paper-like tool correlating between the H₂ penetration depth and it's exposure history, i.e., H₂ pressure and exposure time, is shown in FIG. 8.

1. A composite material-based sensor for the detection of hydrogen presence, comprising:
(i) a H₂ permeable polymeric matrix;
(ii) a reducible organic compound; and
(iii) a catalyst,
wherein the reducible organic compound undergoes a detectable change of color and/or color shade upon hydrogenation.

2. The composite material of claim 1, wherein the change of color is detectable by the naked eye and/or a colorimetric means.

3. The composite material of claim 1, wherein the hydrogenation reaction is irreversible.

4. The composite material of of claim 1, wherein the H₂ permeable polymeric matrix comprises a silicon-containing polymer, a polyurethane polymer, or any combination thereof.

5. The composite material of claim 4, wherein the silicon-containing polymer is a polysiloxane.

6. The composite material of claim 5, wherein the silicon-containing polymer is selected from polydimethylsiloxane (PDMS), polymethylhydrogensiloxane (PMHS), polydiethylsiloxane (PDES), polyphenylmethylsiloxane (PMPS), polydiphenylsiloxane (PDPS), poly(di-n-propylsiloxane), polyditolylsiloxane, polyphenyltolylsiloxane, a polysiloxane having at least one residue selected from a long side chain, branched chain, methoxy-substituted aromatic moiety, cyclic siloxane group or phenylacetylene groups, silalkylene polymers, silarylene polymers, siloxane-silarylene polymers, sesquisiloxane polymers, polysilylenes, polysilazanes and any random, block copolymers or combination thereof.

7. The composite material of claim 6, wherein the silicon-based polymer is PDMS.

8. The composite material of claim 1, wherein the reducible organic compound is a conjugated molecule.

9. The composite material of claim 8, wherein the reducible organic compound is a dye selected from 1,4 bis(phenyl ethynyl)benzene) (PEB), diphenyl butadiyne, diphenyl acetylene, polybutadiene, or polyphenylethynylbenzene.

10. The composite material of claim 1, wherein the catalyst is a transitional metal.

11. The composite material of claim 1, comprising polydimethylsiloxane (PDMS) as the polymeric matrix and 1,4 bis(phenyl ethynyl)benzene) (PEB) as the reducible organic compound.

12. The composite material of claim 11, which, upon reaction with H₂, changes its color shade from bright to dark as seen by the naked eye.

13. The composite material of claim 11, which, upon reaction with H₂, changes its color shade from bright blue to dark blue as seen under UV light.

14. The composite material of claim 13, featuring penetration of color or color shade change in a depth of 200 μm within 1 hour.

15. The composite material of claim 1, in the form of a slab, chip, coating layer, pellet, or tape.

16. A method for detecting or monitoring the presence of hydrogen in an environment containing or suspected of containing free H₂, comprising:
(i) positioning a composite material-based H₂ sensor according claim 1—in the environment containing or suspected of containing free H₂; and
(ii) monitoring changes in the color and/or color shade of the sensor, whereby irreversible stable or progressive color and/or color shade change of the sensor is indicative of hydrogen reaction.

17. The method of claim 16, further comprising the step of determining the hydrogen reaction rate in the composite material-based sensor.

18. A device or article comprising a composite material-based H₂ sensor according to claim 1.

19. The device or article of claim 18, wherein the device or article is coated or in contact with the composite material-based H₂ sensor.

20. The composite material of claim 10, wherein the transitional metal is selected from the group consisting of optionally, Pd, Pt, Ni, Fe, Rh and Ru.
